SEQUENCE LISTING (13 sequences)

SEQ ID NO: 1 - DNA, 84 bp, Artificial
  Multimer of Tcf sites of the COX2 promoter
ggtacccttacccgctacaaagattacccgctacaaagattacccgctacaaagattacccgctacaaagattaccccctcgag

SEQ ID NO: 2 - DNA, 211 bp, Rattus rattus
ctcgaggtctctagggccagccagtgctccagctgaggctgagagagatggcacacagtaggggtgctggagcagcccctccgggaagaggtctggggccatgtcaggacccggcagcctctgattgtgtcctaccctccccttccaggccttcgccccggcagctgcagtcaccaaccacagcatcctttgggtttgacctattgagctc

SEQ ID NO: 3 - DNA, 655 bp, Homo sapiens
gagctcttccttaggcatcagctctctacaattctcacattgagaatatgtgtattttgttagctcaaaccttgttagacatgttaaatgtttagaaatataaatttaacctaccccttgaggtaggtcttgagaggtttgtgagcctaaaaagacatggaggaaccacttattgccacaagcacattgttctaaattatttggaatcagttaattcttccccatctcctacccatgcctgacaccaaagaggagcctctaaatttacagggaatacaaggaagtctactgttctctgctcctctctgggttattagggcacatgggagccctcagttgttttctgctgagcaagagcaaagtccaccttggacttagacagcttgccaaattttttgccagaaggggacctgagttgtgaccactcccagtgtgtgccgggaaaaggctcgtactggtgccagaatctcttactgtcaatgctcccaaaactcaccgcttgcccccaccccttttgcttaaatgacgtggttcttatctcagatcctgatataaagctcctacagctacctggcctgagaagccaactcagactcagccaacaggtaagtgggcattacaggagaagggcgtctctaacatgcactgtagatct

SEQ ID NO: 4 - DNA, 289 bp, Artificial
  Multimer of Tcf sites of the COX2 promoter and fragment comprising between the -287 and -105 base pairs of the nucleosome positioning region of the rat osteocalcin gene promoter
ggtacccttacccgctacaaagattacccgctacaaagattacccgctacaaagattacccgctacaaagattaccccctcgaggtctctagggccagccagtgctccagctgaggctgagagagatggcacacagtaggggtgctggagcagcccctccgggaagaggtctggggccatgtcaggacccggcagcctctgattgtgtcctaccctccccttccaggccttcgccccggcagctgcagtcaccaaccacagcatcctttgggtttgacctattgagctc

SEQ ID NO: 5 - DNA, 26 bp, Artificial
  Forward primer sequence for rat osteocalcin promoter
actcgaggtctctagggccagccagt

SEQ ID NO: 6 - DNA, 27 bp, Artificial
  Reverse primer sequence for rat osteocalcin promoter
cgagctcaggagatgctgccaggacta

SEQ ID NO: 7 - DNA, 36 bp, Artificial
  Forward primer sequence for human REG1A promoter
accatctcgagagtttatcaaatagcttataacttc

SEQ ID NO: 8 - DNA, 35 bp, Artificial
  Reverse primer sequence for human REG1A promoter
tgtatcttcccgaagattttagatctacagtgcat

SEQ ID NO: 9 - DNA, 18 bp, Artificial
  PCR primer
ctagcaaaataggctgtc

SEQ ID NO: 10 - DNA, 19 bp, Artificial
  PCR primer
cgccgggcctttctttatg

SEQ ID NO: 11 - DNA, 6 bp, Artificial
  Canonical Tcf binding site
tcaaag

SEQ ID NO: 12 - DNA, 6 bp, Artificial
  Canonical Tcf binding site
acaaag

SEQ ID NO: 13 - DNA, 5 bp, Artificial
  Consensus sequence of the COX2 gene promoter
caaag

(19) United States
(12) Patent Application Publication
(10) Pub. No.: US 2013/0123346 A1
(43) Pub. Date: May 16, 2013

(54) SYNTHETIC CONSTRUCTS FOR POLYNUCLEOTIDE & PROTEIN EXPRESSION

(71) Applicants: Martin Alejandro Montecino, Santiago (CL); Giancarlo De Ferrari, Santiago (CL); Soraya Bravo, Concepcion (CL); Fernando Cruzat, Concepcion (CL); Osva Ldo Luis Podhajcer, Buenos Aires (AR); Felipe Javier Nunez Hovivera, Ciudad Autonoma De Buene Aire (AR); Eduardo Cafferata, Buenos Aires (AR)

(72) Inventors: Martin Alejandro Montecino, Santiago (CL); Giancarlo De Ferrari, Santiago (CL); Soraya Bravo, Concepcion (CL); Fernando Cruzat, Concepcion (CL); Osva Ldo Luis Podhajcer, Buenos Aires (AR); Felipe Javier Nunez Hovivera, Ciudad Autonoma De Buene Aire (AR); Eduardo Cafferata, Buenos Aires (AR)

(21) Appl. No.: 13/261,451

(22) PCT Filed: Apr. 8, 2011
(86) PCT No.: PCT/IB2011/051534

§ 371 Date: Jan. 18, 2013

Related U.S. Application Data
(60) Provisional application No. 61/322,425, filed on Apr. 9, 2010.

(51) Int. Cl. C12N 15/85; A61K 48/00

(52) U.S. Cl. CPC C12N 15/85; A61K 48/0058
USPC 514/44 R; 435/320.1; 435/325

(57) ABSTRACT

The present invention features, inter alia, nucleic acid constructs that include nucleotide sequences for regulating the expression of a sequence of interest. The sequences in the construct are not naturally associated with one another (i.e., they are heterologous), and they include an enhancer comprising response elements (e.g. Tcf sites) and nucleosome positioning regions. The enhancer can be operably linked to a promoter (e.g., a human REG1A-571 promoter) that drives the expression of a sequence of interest. Also included are vectors comprising these constructs, host cells, kits, pharmaceutical formulations, and methods of treating patients with cancer.

### FIELD OF THE INVENTION

This invention relates to molecular biology and gene therapies. More specifically, the invention relates to a regulatory sequence including at least two heterologous nucleic acid sequences that are functionally linked and that boost the expression of a sequence of interest (e.g., a sequence transcribed to a polynucleotide or translated to a protein of interest).

### BACKGROUND

Response elements or transcription factor-binding sites are DNA sequences that bind transcription factors and activate transcription. Most of them are located within 1 kb from the transcriptional start site.

Transcription factors interact with their response elements using a combination of electrostatic and Van der Waals forces. Hence, most transcription factors bind DNA in a sequence specific manner. However, not all bases in the response element may actually interact with the transcription factor. Thus, transcription factors do not bind just one sequence but are capable of binding a subset of closely related sequences, each with a different strength of interaction. Other constraints, such as DNA accessibility in the cell or availability of cofactors may also help dictate where a transcription factor will actually bind. Thus, given the genome sequence it is still difficult to predict where a transcription factor will actually bind in a living cell. Additional recognition specificity, however, may be obtained through the use of more than one response elements (for example tandem response elements in the same transcription factor or through dimerization of two transcription factors) that bind to two or more adjacent sequences of DNA.

Different response elements participate in several pathways that are active in different conditions or cell types. For example, Tcf sites bind the transcription factor Tcf and are active elements in the Wnt/β-catenin signaling pathway; the cAMP response element (CRE) interacts with CREB (CRE-binding protein), which is regulated by cAMP; estrogen response element (ERE) and glucocorticoid response element (GRE) are the recognition sites of estrogen receptor and glucocorticoid receptor, respectively; heat shock response element (HSE) is present in heat shock protein genes and in response to external stress (e.g. high temperature), the heat shock factor (HSF) interacts with HSE, stimulating expression of heat shock proteins; serum response element (SRE) binds to serum response factor (SRF), which can be activated by many growth factors in serum. Hypoxia response elements (HRE) can enhance the transcriptional activity of a promoter in low oxygen tension conditions. HREs can strengthen the response of a promoter containing estrogen response elements (EREs) in breast tumors (Hernandez-Alcoceba et al., Cancer Gene Therapy 8:298-307, 2001). These same HREs have been combined with radiation response elements (Greco et al., Gene Therapy 9:1403-1411, 2002) and may be a part of a replicative or nonreplicative virus (Ido et al., Cancer Res. 61:3016-3021, 2001). A gene may have many different response elements, allowing complex control to be exerted over the level and rate of transcription.

Tcf sites bind the transcription factor Tcf and are active elements in the Wnt/β-catenin signaling pathway that contributes to pattern formation during the development of many organisms (Byun et al., J. Clin. Pathol. 58:515-519, 2005). This pathway is also active in cancers, cellular growth regulation, motility, and differentiation (Behrens and Lustig, J. Dev. Biol. 48:477-487, 2004). β-catenin activates the Tcf/Lef transcription factors, which regulate the expression of genes that stimulate proliferation and progression through the cell cycle, including c-myc (He et al., Science 281:1509-1512, 1998) and cyclin D1 (Tetsu and McCormick, Nature 398:422-426, 1999). The canonical Tcf binding site is (T/A)CAAAG (SEQ ID NO:11 and SEQ ID NO:12 respectively; Van de Wetering et al., Cell 88:789-799, 1997) and hyperactivation of the Wnt pathway has been studied in gastric cancers (Park et al., Cancer Res. 59:4257-426, 1999; Clements et al., Cancer Res. 62:3503-3506, 2002; Woo et al., Intl. J. Cancer 95:108-113, 2001; Ebert et al., J. Clin. Oncol. 21:1708-1714, 2003; and Ebert et al., Carcinogenesis 23.:87-91, 2002).

Several groups have included Tcf sites in viral vectors. For example, Chen and McCormick (Cancer Res. 61:4445-4449, 2001) built an adenovirus containing the fadd killer gene under the control of multiple Tcf sites (TOP) aiming to target colon cancer cells, in which the Wnt pathway is hyper-activated. Kwong et al. (Oncogene 21:8240-8346, 2002) also used these response elements to selectively express the TK suicide gene in colorectal cancer from an adenoviral vector. Wrighton and others (Lipinski et al., Mol. Ther. 4:365-371, 2001; Lipinski et al., Mol. Ther. 10:150-161, 2004; and Gaedetke et al., Mol. Pharmacol. 4:129-139, 2007) optimized the activity and specificity of a promoter that contains a synthetic TCF multimer by varying the basal promoter, the number of Tcf sites, and the distance between the multimer and the basal promoter. This strategy allowed virtually undetectable expression in normal cells but high levels of expression in cells derived from colon cancer, even when using non-viral transfection. The Iggo group (Brunori et al., J. Virol. 75:2857-2865, 2001; Fuerer and Iggo, Gene Ther. 9:270-281, 2002; Malerba et al., J. Virol. 77:6683-6691, 2003; and Malerba et al., Cancer Gene Ther. 13:273-280, 2006) inserted Tcf site multimers into the P4 viral promoter to direct infection by replicative parvovirus in cells derived from colon cancer. Kuroda et al. (Cancer Res. 66:10127-10135, 2006) created an oncolytic vector from HSV in which replication is conducted by a promoter containing Tcf multimers and whose purpose is to destroy colorectal cancer cells where the Wnt pathway is active. Arber et al. also used Tcf multimers, coupling them to the PUMA suicide gene in cells derived from colon cancer, hepatocellular carcinoma and gastric cancer (see Dvory-Sobol et al., Mol. Cancer. Ther. 5:2861-2871, 2006 and Dvory-Sobol et al., Cancer 109:188-197, 2007).

### SUMMARY

The present invention is based, in part, on our discovery of synthetic constructs that include an enhancer that includes (a) a first sequence comprising multiple copies of a response element, and (b) a second sequence comprising a nucleosome positioning region (e.g., of an osteocalcin (OC) gene promoter). The response element may be one that participates in a pathway that is active in different conditions or cell types. The response element can be selected, for example, but not limited, among Tcf sites, cAMP response elements (CRE), estrogen response elements (ERE), glucocorticoid response elements (GRE), heat shock response elements (HSE), hypoxia response elements (HRE), and serum response elements (SRE).

For example, the response element can be the Tcf site (e.g., a Tcf-binding fragment of a cyclooxygenase-2 (COX-2) gene promoter). To selectively drive gene expression in a particular cell type or tissue type, the synthetic constructs can further include a promoter or a biologically active fragment or other variant thereof that is active in the given cell or tissue. The promoter may also be one that is active in a particular circumstance, such as in a state of cellular stress. For example, the promoter can be one that is active in response to radiation, hypoxia, elevated free radicals, or elevated temperatures. Further, the synthetic constructs can include a sequence that expresses a sequence of interest (e.g., a therapeutic polynucleotide or protein).

One of our objectives was to provide synthetic constructs that enhance the activity of a heterologous promoter and maintain its specificity. The sequence that constitutes the enhancer can be placed upstream or downstream from a promoter that drives the expression of a sequence of interest (e.g., a sequence encoding a therapeutic polynucleotide or a desired protein). The enhancer sequence can also be used to drive viral replication when used, for example, in constructs with the adenoviral E1A gene. The enhancer sequences, the promoter, and the sequence of interest will be arranged in the construct in a manner that allows the sequence of interest to be expressed, preferably at levels higher than would be observed in the absence of the enhancer, and we may therefore describe the enhancer sequences, or the enhancer sequences, the promoter, and the sequence of interest, as being “operably linked.”

As noted, the present constructs include a sequence comprising a response element or, preferably, multiple copies of a response element (e.g., about two to about 10 copies) of such an element. In a preferred embodiment the response element is the Tcf site or, preferably, multiple copies of the Tcf site (e.g., about two to about 10 copies) of such a site. Thus, the constructs can include a portion of the regulatory region of a COX-2 gene, as this region includes Tcf-binding sites. More specifically, the constructs can include SEQ ID NO:1:

(SEQ ID NO: 1)
GGTACCCTTACCCGCTACAAAGATTACCCGCTACAAAGATTACCCGCTAC
AAAGATTACCCGCTACAAAGATTACCCCCTCGAG.

The constructs can also include other “first” sequences that include Tcf-binding sites, including biologically active fragments or other variants of SEQ ID NO:1. For example, the first sequence can be nucleotides 7-78 of SEQ ID NO:1. The first six nucleotides and the last six nucleotides can be absent or may vary (i.e., may be sites recognized by other restriction endonucleases), as these represent restriction sites to facilitate cloning (in SEQ ID NO:1, sites for KpnI and XhoI, respectively). The variable restriction sites are underlined and the Tcf-binding sites appear in bold-faced type and are underlined.

As noted, the enhancer can also include a second sequence that includes a nucleosome positioning region, such as a region of an osteocalcin gene promoter. For example, a construct can include the sequence extending from −287 to −105 (relative to the transcriptional start site) of the rat osteocalcin gene promoter. This sequence, corresponding to the nucleotides from −287 to −105 is within SEQ ID NO:2:

(SEQ ID NO: 2)
CTCGAGGTCTCTAGGGCCAGCCAGTGCTCCAGCTGAGGCTGAGAGAGATG
GCACACAGTAGGGGTGCTGGAGCAGCCCCTCCGGGAAGAGGTCTGGGGCC
ATGTCAGGACCCGGCAGCCTCTGATTGTGTCCTACCCTCCCCTTCCAGGC
CTTCGCCCCGGCAGCTGCAGTCACCAACCACAGCATCCTTTGGGTTTGAC
CTATTGAGCTC

The first six nucleotides and the last six nucleotides represent restriction sites, which can be excised if desired or modified to facilitate cloning.

The first and second sequences in the enhancer can be operably linked to any promoter to facilitate expression of a downstream sequence of interest. Where cell type-specific or tissue-specific expression is desired, the promoter can be one that is suitably active in a given cell type or tissue. For example, the promoter can be one that normally drives the expression of a downstream sequence in a tumor cell. While the sequence of interest can be a naturally occurring sequence (e.g., an entire gene or a fragment thereof), it can also be a non-naturally occurring sequence (e.g., a mutant of a gene or a fragment thereof). We may refer to fragments and other variants of a naturally occurring sequence as “biologically active” where they function to a useful extent. For example, where the activity of a mutant of a sequence of interest is comparable to the function of the wild type counterpart, the mutant is biologically active. Similarly, where the activity of a fragment or other variant of the enhancer or promoter sequence is comparable to the function of its wild type counterpart, the fragment or variant sequence is biologically active. Non-naturally occurring sequences can differ from their wild type counterparts by virtue of an insertion, substitution or deletion of one or more nucleotides, and the extent of the difference can be described in terms of a “percent identity.” For example, a fragment or other variant can be at least or about 80% (e.g., at least or about 80%, 85%, 90%, or 95%) identical to its wild type counterpart.

The constructs can be plasmid or viral vectors, and either type of vector can include the enhancer sequences described herein, with or without additional regulatory elements (i.e., a promoter) and a sequence of interest (e.g., a sequence producing an RNA or protein of interest).

The invention also provides methods for expressing nucleic acids in a host cell. These methods can be carried out using recombinant DNA or recombinant viral vectors that include the enhancer sequences described herein (e.g., the response element and the nucleosome positioning region represented by SEQ ID NO:2), a promoter, and a sequence of interest. Transcribed RNA or encoded protein can subsequently be isolated or purified from the host cells if desired.

The invention also provides methods of treating a patient who needs gene therapy. These methods can be carried out by administering to the patient an effective amount of a pharmaceutical composition containing a construct as described herein. For example, the construct can include an enhancer sequence as described herein, a promoter that is active in the type of cell where the therapy is needed in the patient, and a sequence of interest that encodes a therapeutic polynucleotide or protein sequence. In one embodiment, the methods are methods of treating a patient who is suffering from cancer. These methods can be carried out by administering to the patient an effective amount of a pharmaceutical composition containing a construct as described herein. For example, the construct can include an enhancer sequence as described herein, a promoter that is active in the type of cell that is proliferating uncontrollably in the patient, and a sequence of interest that encodes a therapeutic polynucleotide or protein sequence (e.g., a toxin) that kills the tumor cells or slows or arrests their growth.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a diagram illustrating the structure of a construct of the present invention. RE represents a response element, NPS represents a nucleosome positioning sequence, TSP represents a tissue or tumor specific promoter, and SI represents a sequence of interest.

FIG. 2 is a series of bar graphs representing data obtained from studies of constructs driving luciferase expression in two human gastric tumor cell lines: MKN-45 (kindly provided by Dr. Andrew Quest; Universidad de Chile, Santiago, Chile) and AGS (from the American Type Culture Collection (ATCC) # CRL-1793), WI-38, and HEK293. As controls, expression was assessed in WI-38 cells (ATCC # CCL-75; human lung fibroblasts) and HEK293 cells derived from human embryo kidney (ATCC # CRL-1573). One construct included a pART vector with enhancer sequences and a region of the REG1A promoter (pGL3pART) and one construct included vector with the REG1A-571 promoter alone (pGL3Reg1A-571). Luciferase enzyme activity was assessed and is presented in RLUs (relative luciferase units). (*=p<0.05; **=p<0.01; ***=p<0.001. ANOVA test.)

FIG. 3 is a bar graph showing an increase in luciferase activity in AGS cells transformed with increasing amounts (100 μl, 200 μl, and 400 μl) of an adenovirus in which a luciferase-encoding sequence is placed under the control of a pART synthetic enhancer with the REG1A promoter. (***=p<0.001. ANOVA test.)

FIG. 4 is a bar graph showing luciferase activity in different conditions for different plasmids in SK-N—SH cell line (ATCC # HTB-11) from human neuroblastoma, showing that pART with Tcf sites increase luciferase activity in a cell line where the Wnt pathway is not naturally active. (**=p<0.01. ANOVA test.)

### DETAILED DESCRIPTION

The present invention features nucleic acid constructs that include an enhancer having a first sequence that includes at least one copy of a response element and a second sequence that includes at least one nucleosome positioning region.

The response element can be any that is participating in an active pathway in the cell type or tissue where the construct will act. The response element can be selected, for example, but not limited, among Tcf sites, cAMP response elements (CRE), estrogen response elements (ERE), glucocorticoid response elements (GRE), heat shock response elements (HSE), hypoxia response elements (HRE), and serum response elements (SRE).

In a preferred embodiment the first sequence includes at least one copy of a Tcf site. The Tcf site can be as described herein and is naturally present in the regulatory region of the COX-2 gene.

The COX-2 gene has been described as a target of the Wnt pathway (Howe et al., J. Biol. Chem. 276:20108-20115, 2001; Araki et al., Cancer Res. 63:728-734, 2003; Castellone et al., Science 310:1504-1510, 2005; Eisinger et al., J. Biot. Chem. 281:2074-2082, 2006). COX-2 is involved in the repair of peptic ulcers and colitis, as well as in tumor development and progression, angiogenesis, and inhibition of apoptosis (Chen et al., World J. Gastroenterol. 11:1228-1231, 2005). The overexpression of COX-2 in gastric cancer has been well-studied (Han et al., Digestive Surg. 20:107-114, 2002; Saukkonen et al., APMIS 111:915-925, 2003; Yasuda et al., J. Gastroenterol. 40:690-697, 2005; Oshima et al., The EMBO J. 23:1669-1678, 2004; and Konturek et al., Regulatory Peptides 93:13-19, 2000). We have characterized a new Tcf site in the promoter of the human COX-2 gene. This Tcf site spans the sequence from −692 bp to −683 bp (with a core element at −689/−684) of the human COX-2 gene promoter and, although it contains the core elements of the consensus sequence (CAAAG; SEQ ID NO:13), it is different from the TCF sites used by other groups to construct therapeutic vectors as it includes different flanking nucleotides. We used this sequence to create a multimer having four copies of the Tcf site, which can be used as the “first” sequence of the enhancer in the present constructs.

The “second” sequence of the enhancer includes a nucleosome positioning region. The formation of chromatin (DNA packaged around histones) represents an important and powerful mechanism to regulate gene expression (Hebbar and Archer, J. Biol. Chem. 283:4595-4601, 2008). The key structural element in chromatin is the nucleosome, which is formed by an octamer of histone proteins wrapped by 147 bp of DNA (Zhang et al., BCM Genomics 9:1-11, 2008). Identifying the precise positions of nucleosomes in the regions that control the expression of a given gene is important because it allows one to predict the mechanisms by which the gene is regulated in a cell (Rando and Ahmad, Curr. Opin. In Cell Biol. 19:250-256, 2007). Montecino et al. have identified a DNA region capable of positioning a nucleosome in the promoter of the rat osteocalcin gene (Gutiérrez et al., J. Biol. Chem. 282:9445-9547, 2007). We reasoned that, since a nucleosome can help determine the three dimensional organization of a promoter region and help coordinate the action of different transcription factors associated with the promoter, a nucleosome positioning sequence (e.g., the nucleosome positioning sequence of an osteocalcin gene) would be useful as a second enhancer element in the present constructs. The particular sequence we incorporated in the studies described below extends from −287 to −105 (with respect to the transcription start site) of the rat osteocalcin (OC) gene promoter.

Where the present constructs include a response element and a nucleosome positioning region of a gene, the construct will constitute a synthetic enhancer for the expression of genes in particular tissues, but we do not expect it will have promoter activity per se. Instead, we expect the synthetic enhancer to improve the function of operably linked promoters, including tumor-specific or tissue-specific promoters. Therefore, the present constructs, with any of the enhancers described herein, can further include all of a tissue-specific or cell type-specific promoter or a fragment or other variant thereof that confers expression, including selective expression. Non-limiting examples of the promoter include a region of the promoter of REG1A, which is selectively active in gastric cancer cells, and other promoters like A33, which exhibit high activity in colon tumor cells. Other promoters useful in the present constructs are known in the art. For example, the promoter can be a tissue-specific promoter as shown in Table 1, below, or a tumor-specific promoter as shown in Table 2, below. These Tables are reproduced from Robson and Hirst (J. Biomed. Biotechnol. 2:110-137, 2003). The types of tumors referenced can be treated with the constructs described herein when those constructs express a therapeutic polynucleotide or protein.

TABLE 1
Tissue-specific promoters used in cancer gene therapy.

| Promoter | Target tissue/tumour |
|---|---|
| Tyrosinase | Melanocytes/melanoma |
| Prostate-specific antigen (PSA) | Prostate |
| Prostate-specific membrane antigen | Prostate/also targets vascular |
| (PSMA) | endothelium of other tumors |
| Probasin | Prostate |
| Human glandular kallikrein (hK2) | Prostate |
| Glial fibrillary acidic protein (GFAP) | Glial/glioma |
| Myelin basic protein (MBP) | Glial and astrocytes/glioma |
| Myelin proteolipid protein | Glial/glioma |
| Neural specific enolase | Neuronal/SCLC |
| Neuronal specific synapsin I | Neuronal |
| Ncx/Hox IILI | Neural crest derived cells/ |
| | neuroblastoma |
| Albumin | Liver/hepatocellular carcinoma |
| Surfactant protein B | Type II alveolar and bronchial |
| | cells/lung cancer |
| Thyroglobulin | Thyroid/thyroid carcinomas |
| Ovarian-specific promoter | Ovarian |

TABLE 2
Tumour-specific promoters.

| Promoter | Tumour target |
|---|---|
| Telomerase | Lung, colon, ovarian, bladder, cervical, liver, |
| | glioma |
| CEA | Colorectal, pancreatic, cholangiocarcinoma, |
| | breast, lung |
| Alpha feto protein (AFP) | Hepatoma |
| Erb B2 | Breast, pancreatic, ovarian |
| DF3/MUC1 | Breast, choloangiocarcinoma |
| Osteocalcin | Prostate, ovary, lung, brain, osteoblasts |
| L-plastin | Ovarian, breast, fibrosarcoma |
| Midkine | Embryonal carcinoma; Wilm's tumours, |
| | neuroblastoma, pancreatic, oespohageal |
| Secretory leukoprotease | Lung, breast, oropharyngeal, bladder, |
| inhibitor (SLP1) | endometrial, ovarian, colorectal, cervical |
| Alpha lactalbumin | Breast |
| Myc-max | Breast, lung |
| Somatostatin | Malignant melanoma of soft parts |
| Cox2 | Ovarian, pancreatic, gastrointestinal |
| Ornithine decarboxylase | Colon and neuroblastoma |
| Epithelial glyocoprotein 2 | Carcinomas |
| (EPG2) | |
| c-Myb-responsive | Hematopoietic tumours |
| promoters | |
| Gastrin-releasing peptide | Lung |
| Metallothionein | Ovarian |
| Calponin | Soft tissue and bone tumours |
| H19 | Bladder |
| Tcf | Colon |
| Calretinin | Mesathelioma |
| Calcitonin/calcitonin | Thyroid/thyroid medullary cancer |
| gene-related peptide | |

Cell cycle-related

| | |
|---|---|
| CyclinA | Melanoma |
| Endoglin | Endothelial cells |
| IGF-1-R | Tumours mutant for p53, cMyb or |
| | EWS/WT1 |
| E2F-1 | Glioma |

The expression of REG1A in human gastric tissue was first reported in 1990 (Watanabe et al., J. Biol. Chem. 381:397-403, 2004). In this work, Northern blot analysis revealed messenger RNA in a sample of human gastric mucosa. Since then, studies have shown that REG1A expression is restricted to endocrine gastric cells and epithelial cells of the Chief type (Ashcroft et al., Biochem. J. 381:397-403, 2004 and Yoshino et al., Am. J. Gastroenterol. 100:2157-2166, 2005). Further, expression is associated with inflammation and injury (Yoshino et al., Am. J. Gastroenterol. 100:2157-2166, 2005; Fukui et al., Gastroenterol. 115: 1483-1493, 1998; Masamune et al., Gastroenterol. 116; 1330-1342, 1999; Akiyama et al., Proc. Natl. Acad. Sci. USA 98:48-53, 2001; Fukui et al., Laboratory Invest. 83:1777-1786, 2003; Kiyaoka et al., Oncogene 23:3572-3579, 2004; Fukui et al., Digestion 69:177-184, 2004; Judd et al., Gastroenterol. 126:196-207, 2004; Sekikawa et al., Gut 54:1437-1444, 2005; Sekikawa et al., Gastroenterol. 128:642-653, 2005; Steele et al., Am. J. Physio. Gastrointest. Physiol. 293:G347-G354, 2007; Takaishi and Wang, Cancer Sci. 98:284-293, 2007; Sekikawa et al., Carcinogenesis 29:76-83, 2008; and Lee et al., Cancer Res. 68:3540-3548, 2008). The overexpression of REG1A in gastric tumor tissue and tumor cell lines derived from gastric cancers has been widely documented (Sekikawa et al., Gastroenterol. 128:642-653, 2005; Sekikawa et al., Carcinogenesis 29:76-83, 2008; Lee et al., Cancer Res. 68:3540-3548, 2008; Kumar et al., Cancer 100:1130-1136, 2004; Ose et al., Oncogene 26:349-359, 2007; and Kazumori et al., Gastroenterol. 119:1610-1622, 2000). Studies have also established that REG1A is an essential growth factor for the regeneration of the gastric mucosa (Fukui et al., Digestion 69:177-184, 2004 and Ose et al., Oncogene 26:349-359, 2007) and that an increase in REG1A expression in tumor cells is associated with cellular proliferation (Yoshino et al., Am. J. Gastroenterol. 100:2157-2166, 2005; Fukui et al., Gastroenterol. 115:1483-1493, 1998; and Kazumori et al., Gastroenterol. 119:1610-1622, 2000).

A fragment containing 1.096 bp of the REG1A promoter has been used in prior reporter gene studies to evaluate the promoter response to IL-8 (Yoshino et al., Am. J. Gastroenterol. 100:2157-2166, 2005). In 2005, Chiba and collaborators described regulation by INFy and IL-6 in cells derived from colon cancer, using a 1.195 bp region of the REG1A promoter (Sekikawa et al., Gut 54:1437-1444, 2005). It has also been shown by means of functional tests using various deletions from the human REG1A promoter and site-directed mutagenesis that the regulation by IL-6 in MKN-74 cells is dependent on the STAT3 factor signaling pathway (Sekikawa et al., Carcinogenesis 29:76-83, 2008). At around the same time, Perret and collaborators proposed that transcriptional regulation of the REG1A gene is modulated through the Wnt/β-catenin signaling pathway (Cavard et al., Oncogene 25:599-608, 2006). However, their work did not include functional tests with the REG1A promoter.

In other studies, Hervé reported the construction of an adenovirus containing the NIS gene under the control of the RegIII alpha gene promoter, which conferred transcriptional ability and tumor selectivity to this vector and helped optimize radiotherapy against liver cancer (Herve et al., Human Gene Therapy 19:915-926, 2008). To our knowledge, there are no additional reports related to the use of REG promoters to direct the expression of therapeutic genes or to direct replication of recombinant vectors.

We have demonstrated that the fragment of the human REG1A gene promoter that extends from −571 to +75 with respect to the transcriptional start site is sufficient and necessary to drive the expression of a gene of interest in a selective manner in gastric tumor cells.

As shown in the data below, Tcf sites enhance promoter activity in response to the Wnt signaling pathway and the nucleosome positioning region allows the DNA of synthetic promoters to acquire a three dimensional structure that facilitates the functional interaction between distal and proximal regulatory complexes. Furthermore, the promoter can provide selectivity (e.g., selective expression in gastric cancer).

The nucleotide sequences used in the present constructs may be described as “isolated”, which means they have been separated from the nucleotides sequences with which they are naturally associated or purified from other sequences in a cell or organism where they are naturally present. “Isolated” nucleotide sequences can be those that are obtained through standard purification techniques as well as sequences prepared using recombinant technologies or chemical synthesis (e.g., by the PCR).

A “variant” of a nucleotide sequence is a nucleotide sequence that is different from a sequence disclosed herein and/or different from a naturally occurring sequence. The variant may be obtained through modifications such as insertion, substitution or deletion of one or more nucleotides.

“Tissue-specific” expression encompasses highly correlated expression, but not necessarily exclusive expression.

We may describe various parts of the present constructs as “heterologous”, meaning that the one part is different from another part and the two parts are not naturally associated with one another. For example, a promoter and a sequence of interest are heterologous when the promoter normally drives the expression of a different sequence of interest.

In general, a “therapeutic” sequence of interest (e.g., a gene) is a nucleotide sequence (e.g., a DNA sequence) that produces a polynucleotide or encodes an amino acid that directly or indirectly confers a positive therapeutic effect on a host cell in which it is expressed and can, in turn, improve the clinical outlook for a patient. In accordance with this invention, the host cells can be tumor cells.

An exemplary enhancer is represented by SEQ ID NO:4.

GGTACCCTTACCCGCTACAAAGATTACCCGCTACAAAGATTACCCGCTAC
AAAGATTACCCGCTACAAAGATTACCCCCTCGAGGTCTCTAGGGCCAGCC
AGTGCTCCAGCTGAGGCTGAGAGAGATGGCACACAGTAGGGGTGCTGGAG
CAGCCCCTCCGGGAAGAGGTCTGGGGCCATGTCAGGACCCGGCAGCCTCT
GATTGTGTCCTACCCTCCCCTTCCAGGCCTTCGCCCCGGCAGCTGCAGTC
ACCAACCACAGCATCCTTTGGGTTTGACCTATTGAGCTC

The aforementioned sequence comprises four Tcf sites of the COX promoter and the nucleosome positioning region of the rat osteocalcin gene promoter.

As noted, the response element of the enhancer can vary and can be selected among any that participate in an active pathway in the cell type or tissue where the enhancer will act. The response element can be selected, for example, but not limited, among Tcf sites, cAMP response elements (CRE), estrogen response elements (ERE), glucocorticoid response elements (GRE), heat shock response elements (HSE), hypoxia response elements (FIRE), and serum response elements (SRE). The enhancer can be placed upstream or downstream from a specific promoter sequence that drives expression of a sequence of interest. The level of expression is heightened when the promoter is operably linked to an enhancer as described herein. As noted, the promoter can vary and can be selected based on its ability to drive expression of a sequence of interest in a particular cell type or under particular conditions. For example, the present constructs can include a hypoxia response element (HRE) that enhances the transcriptional activity of a promoter or response element in low oxygen tension conditions. HREs can strengthen the response of a promoter containing estrogen response elements (EREs) in breast tumors (Hernandez-Alcoceba et al., Cancer Gene Therapy 8:298-307, 2001). These same HREs have been combined with radiation response elements (Greco et al., Gene Therapy 9:1403-1411, 2002) and may be a part of a replicative or nonreplicative virus (Ido et al., Cancer Res. 61:3016-3021, 2001). The promoters or other regulatory sequences of the present constructs can include HREs, EREs, or both.

The present constructs can be used to express essentially any sequence of interest, including any gene (e.g., a naturally occurring gene) or any desired RNA (e.g., an RNA that mediates RNAi). The constructs can also include sequences of interest that encode non-naturally occurring peptides or proteins. Generally, the term “peptide” is used to describe shorter amino acid polymers, and the term “protein” is used to describe longer amino acid polymers, such as full-length, naturally occurring proteins. Both, however, are chains of amino acid residues, and either may also be referred to as a “polypeptide”.

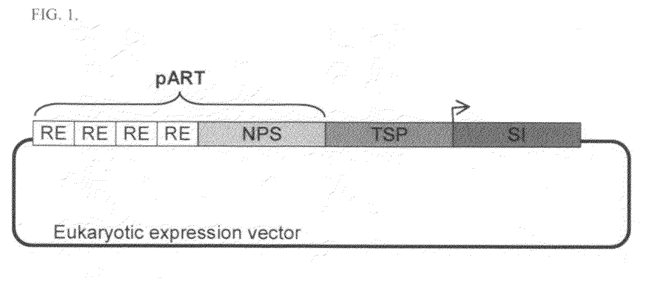

FIG. 1 represents a diagram of the construct structure with the response elements, the nucleosome positioning sequence, the tissue or tumor specific promoter and the sequence of interest.

Suitable host cells are well known in the art, as are methods of introducing plasmid and/or viral vector constructs generated by recombinant methods into a host cell, culturing the host cell to allow for expression from a sequence of interest, and isolating or purifying the transcribed or translated product. DNA can be introduced into a host cell using any construct capable of replicating inside the cell. For example, the construct can be, without limitation, a plasmid, a DNA virus, a retrovirus, or an isolated nucleotide molecule. Transfer can be mediated by methods known in the art, including electroporation and lipofection.

Useful DNA viruses include adenoviruses. More than 40 serotypes of human adenoviruses are well-known, and the Ad5 adenovirus may be particularly preferred as a viral vector for use with the present constructs. However, modified capsid and/or fiber Ad5 adenoviruses, such as the capsid of the Ad3 adenovirus or fiber modification with a RGD motif can also be used.

The present constructs can be made using standard recombinant techniques, which are well known in the art. For example, DNA can be cleaved at specific sites using restriction enzymes and ligated to previously non-associated sequences. In general, the results can be verified with electrophoretic separation in agarose gels. The vector:insert ratios can vary from 1:1 to 1:4, estimating the ratio between the fragments with the following formula:

$$( \frac{ng vector \times Kb insert}{Kb vector} ) \times ( \frac{insert Ratio}{vector} ) = ng of insert$$

The constructs or vectors generated in this invention may be administered to a patient through injection, oral or topical administration, using an adequate carrier as a vehicle. Adequate carriers may be aqueous, lipidic, and liposomal, among others.

This invention is illustrated below through detailed experimental examples. The purpose of these examples is to provide a better understanding of the invention, although these examples should not, by any means, be considered as limitations to the invention.

### EXAMPLES

### Example 1

### Construction of the pART Synthetic Enhancer Operably Linked to a REG1A-571 Specific Promoter

An oligonucleotide containing four Tcf sites, corresponding to the sequence that extends from nucleotides −692 to −683 (core element: −689/−684) in the COX-2 promoter, was obtained through chemical synthesis (see SEQ ID NO:1) and incorporated into pART at the KpnI and XhoI restriction sites (New England Biolabs, Ipswich, Mass., USA).

The nucleosome positioning region was obtained from the rat osteocalcin (OC) promoter using PCR and the specific primers: ACTCGAGGTCTCTAGGGCCAGCCAGT (forward) and CGAGCTCAGGAGATGCTGCCAGGACTA (reverse) (SEQ ID NO:5 and SEQ ID NO:6, respectively). The resulting 182 base pair fragment was incorporated into pART at the XhoI and Sad restriction sites (New England Biolabs, Ipswich, Mass., USA). The underlined sequence represents the restriction enzymes sites.

A fragment of the human REG1A gene promoter was amplified by the PCR technique from a sample of human genomic DNA using the specific primers: ACCATCTCGAGAGTTTATCAAATAGCTTATAACTTC (forward) and TGTATCTTCCCGAAGATTTTAGATCTACAGTGCAT (reverse) (SEQ ID NO:7 and SEQ ID NO:8, respectively). The PCR product thus obtained was introduced directly into the pGL3-Basic vector (Promega Corp., Madison, Wis., USA) through site-directed cloning using the KpnI and BglII restriction enzymes (New England Biolabs, Ipswich, Mass., USA). A fragment corresponding to the −571/+75 base pairs region of the human REG1A promoter (SEQ ID NO:3) was also obtained from this construct, through cleavage with the HindIII and BglII restriction enzymes (New England Biolabs, Ipswich, Mass., USA).

SEQ ID NO: 3:
GAGCTCTTCCTTAGGCATCAGCTCTCTACAATTCTCACATTGAGAATATG
TGTATTTTGTTAGCTCAAACCTTGTTAGACATGTTAAATGTTTAGAAATA
TAAATTTAACCTACCCCTTGAGGTAGGTCTTGAGAGGTTTGTGAGCCTAA
AAAGACATGGAGGAACCACTTATTGCCACAAGCACATTGTTCTAAATTAT
TTGGAATCAGTTAATTCTTCCCCATCTCCTACCCATGCCTGACACCAAAG
AGGAGCCTCTAAATTTACAGGGAATACAAGGAAGTCTACTGTTCTCTGCT
CCTCTCTGGGTTATTAGGGCACATGGGAGCCCTCAGTTGTTTTCTGCTGA
GCAAGAGCAAAGTCCACCTTGGACTTAGACAGCTTGCCAAATTTTTTGCC
AGAAGGGGACCTGAGTTGTGACCACTCCCAGTGTGTGCCGGGAAAAGGCT
CGTACTGGTGCCAGAATCTCTTACTGTCAATGCTCCCAAAACTCACCGCT
TGCCCCCACCCCTTTTGCTTAAATGACGTGGTTCTTATCTCAGATCCTGA
TATAAAGCTCCTACAGCTACCTGGCCTGAGAAGCCAACTCAGACTCAGCC
AACAGGTAAGTGGGCATTACAGGAGAAGGGCGTCTCTAACATGCACTGTA
GATCT

### Example 2

### Construction of the pART-REG1A-LUC Construct

The three sequences obtained as described in Example 1 were ligated using the DNA ligase of the T4 bacteriophage, following the supplier's protocols (New England Biolabs Inc., Beverly Mass., USA). The cassette obtained (including SEQ ID NO: 1, SEQ ID NO:2, and SEQ ID NO:3) was introduced into the pGL3 vector at the KpnI and BglII restriction sites (New England Biolabs Inc., Beverly Mass., USA) using the same ligase mentioned above. Cloning was checked through a digestion test with restriction enzymes and confirmed by automatic sequencing using the following primers: CTAGCAAAATAGGCTGTC(PGL3FWD) (SEQ ID NO:9) and CGCCGGGCCTTTCTTTATG (LUC-compl) (SEQ ID NO:10).

The pGL3-Basic plasmid contains the modified Firefly luciferase reporter gene (luc+) that prevents the union of gene regulatory factors, eliminates undesirable restriction sites, prevents luciferase protein transport to peroxisomes, and includes a Kozak sequence in the 5′ end of the luciferase gene in order to optimize translation efficiency.

### Example 3

### Expression of Luciferase Driven by pART-REG1A in Different Cell Lines

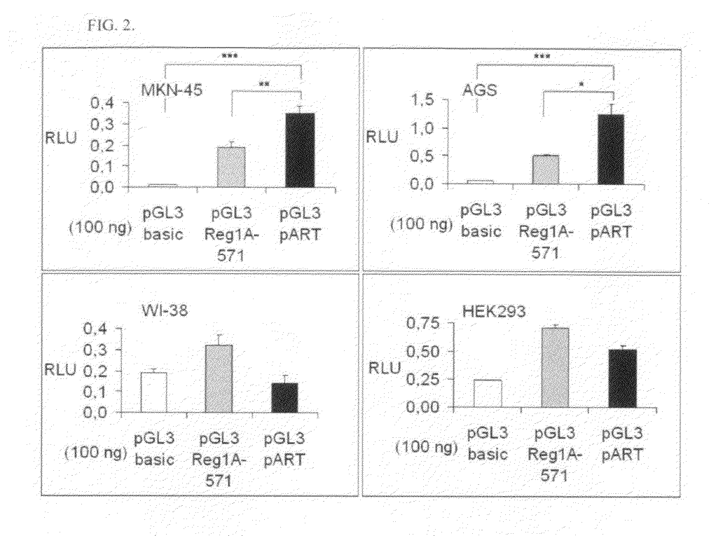

The presence of the luciferase reporter gene allowed us to quantify the activity of the pART synthetic promoter by measuring luciferase enzyme activity. The results of at least three independent experiments, each one of them measured in triplicate, are shown in FIG. 2. The measurements were made using the AGS and MKN-45 cell lines, which represent advanced stages of gastric cancer, in HEK 293 cells derived from human embryonic kidney, and in WI-38 cells derived from human lung (a nontransformed stage).

The cells were seeded in 24-well plates at a density of 3−5×10⁴ cells per well. After 24 hours, cells were transfected using the FuGENE 6 Transfection Reagent (Roche) following the supplier's instructions. Each treatment was conducted in triplicate, in at least three independent experiments, incubating 100 ng of treatment plasmid with 2 ng of pRL-SV40 for 5 minutes with 50 μl of medium without serum, and, in parallel, 2 μA of FuGENE with 50 μl of the same medium. These two preparations were mixed and incubated for 20 minutes at room temperature. The transfection was performed on the cells in 400 μl fresh medium with 10% serum. The cells were incubated for 24 hours at 37° C. with 5% CO₂.

We used the Dual Luciferase Reporter Assay System (Promega Corp., Madison, Wis., USA) for the luciferase test. This system implies the simultaneous expression of two individual reporter enzymes in a same system, making it possible to evaluate the activity produced by the luciferase enzymes Firefly (Photinus pyralis) and Renilla (Renilla reniformis) in only one sequential test. The enzymatic activity was determined in a Victor 3 (Perkin-Elmer, Finland) luminiscence reader. Data were standardized as follows:

$$\frac{Firefly Luciferase Units}{Renilla Luciferase Units} = Relative Luciferase Units ( RLU )$$

The data were expressed as the induction values relative to the activity obtained with the pGL3-Basic control plasmid (without promoter).

As shown in FIG. 2, the pART-REG1A construct produces comparable activity in the MKN-45 and AGS cellular lines, where maximum activity is 30 and 23 times higher than produced the empty vector, respectively. On the other hand, in the control WI-38 cell line and in the HEK 293 tumor cell line derived from human kidney, the pART promoter is virtually inactive. The selective activity of the pART-REG1A promoter fragment in the cell lines derived from advanced gastric cancer indicates that this promoter can be used to direct the expression of heterologous genes of interest in gastric cancer cells. (*=p<0.05; **=p<0.01; ***=p<0.001. ANOVA test.)

### Example 4

### Construction of the Ad-pART-REG1A-LUC Adenoviral Vector

For the construction of the Ad-pART-REG1A-LUC adenoviral vector, we subcloned the pART-REG1A promoter into the shuttle pDC-Luciferase plasmid kindly donated by Doctor Sergio Oñate (Department of Physiopathology, Biological Sciences School, Universidad de Concepción). This shuttle plasmid contains a part of the type 5 human adenovirus, a multicloning site, and the luciferase reporting gene, in that order. Subcloning was accomplished by extracting the pART promoter from the pGL3-pART vector with KpnI (New England Biolabs) and HindIII (New England Biolabs), filling the KpnI overhang with Klenow fragment (New England Biolabs), subjecting the pDC-luciferase vector to cleavage with Accl (New England Biolabs) and HindIII, filling the AccI overhang with Klenow fragment, and finally ligating both sequences with T4 ligase (New England Biolabs). The subcloning was confirmed by automatic DNA sequencing using the primer LUC-compl: CGCCGGGCCTTTCTTTATG (SEQ ID NO:10).

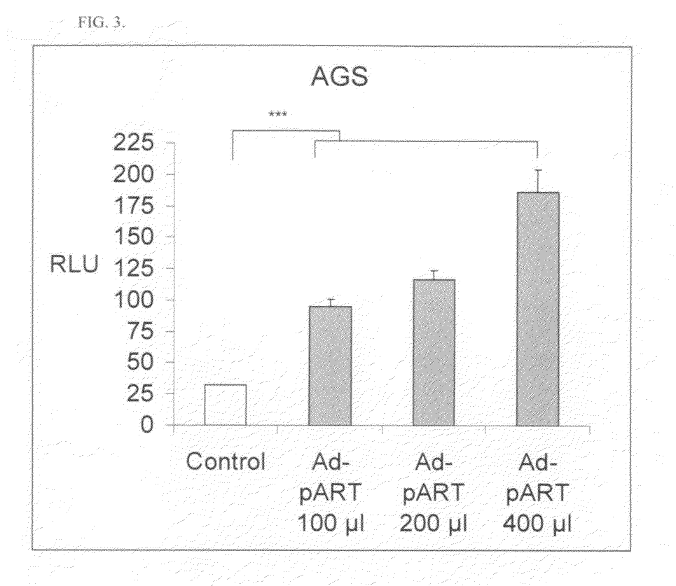

The shuttle plasmid containing pART-REG1A was cotransfected with the pBHGlox plasmid (Microbix Biosystems) into HEK293QBI cells (kindly donated by Dr. Sergio Lavanderos, Faculty of Chemical and Pharmaceutical Sciences, Universidad de Chile) at a ratio of 5:1. These cells are derived from a human embryonic kidney cell line and express the Elgene from the type 5 human adenovirus genome. The cells were cultured until they reached a 50% confluence in D-MEM 5% medium (GIBCO). The cotransfection was carried out using Lipofectamine (Invitrogen). By means of homologous recombination between the adenoviral DNA samples cotransfected, we obtained recombinant adenoviruses that express the exogenous luciferase protein under the control of the pART promoter, denominated Ad-pART-REG1A-LUC. Ad-pART-REG1A-LUC was amplified and tested for luciferase activity following infection in the AGS and MKN-45 cell lines, which are derived from gastric cancer. As shown in FIG. 3, luciferase activity (controlled by total protein quantity) is observed after infection of AGS gastric tumor cells with varying amounts of Ad-pART-REG1A-LUC. (***=p<0.001. ANOVA test.) These results are representative of at least three independent experiments, performed in both AGS and MKN-45 cells.

### Example 5

### Expression of Luciferase in SK-N—SH Cell Line with Different Constructs in Different Conditions

The effect of pART as enhancer in a cell line where the Wnt pathway is not active was assayed.

Cells from SK-N—SH cell line (ATCC # HTB-11) from human neuroblastoma were transfected with the following plasmids:

pGL3 basic: pGL3-Basic vector (Promega Corp., Madison, Wis., USA);

pGL3-NSE: pGL3 further comprising enolase promoter (Addgene plasmid 11606);

pGL3-pART-NSE: constructed according to standard techniques well known in the art, inserting pART upstream of the enolase promoter in pGL3-NSE, using the restriction enzyme NheI and the DNA ligase T4; and

STF: plasmid used as positive control that has Super Top Flash artificial promoter that responds to Wnt pathway.

Another sample of cells cotransfecting with β-catenin was prepared for each plasmid in order to exogenously activate Wnt pathway.

Activity of luciferase was measured as described in Example 3.

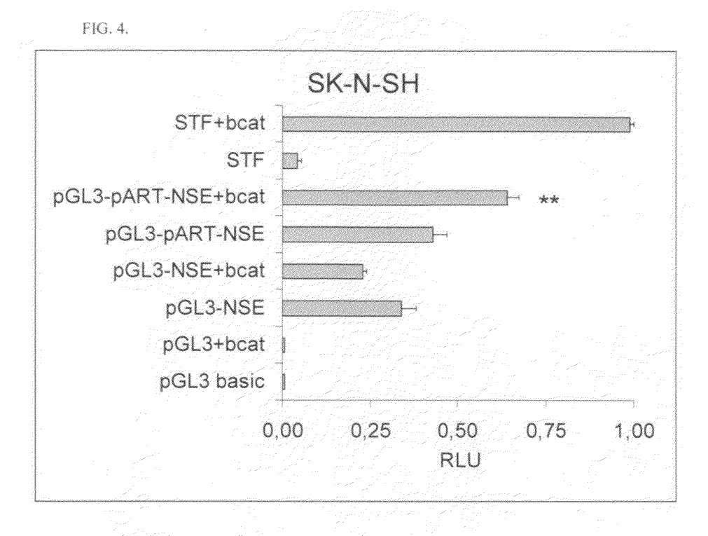

FIG. 4 shows luciferase activity in SK-N—SH cell line with each plasmid with and without β-catenin. (**=p<0.01. ANOVA test.)

Results show that pGL3-pART-NSE has a base activity compared with pGL3-NSE, and when Wnt pathway is activated with β-catenin, the activity in pGL3-pART-NSE+bcat is increased. This result shows that pART is also effective in cell lines different from tumor cell lines, where Wnt pathway is not naturally active. This implies that other response elements can be use instead of Tcf sites. The results also show that other promoters, as enolase promoter, different from tumor specific promoters can be activated by pART.

While it is evident that the working examples described above support the invention presented herein, it is also evident that numerous modifications and other variations are possible and will be evident to one of ordinary skill in the art.

Accordingly, other embodiments are within the scope of the following claims:

What is claimed is:

1. A nucleic acid construct comprising an enhancer, wherein the enhancer comprises a first sequence having at least one response element and a second sequence having at least one nucleosome positioning region.

2. The construct of claim 1, wherein the first sequence comprises at least one Tcf site.

3. The construct of claim 2, wherein the first sequence comprises 2-10 Tcf sites.

4. The construct of claim 3, wherein the first sequence comprises four Tcf sites.

5. The construct of any of claims 1-4, wherein the first sequence comprises SEQ ID NO:1 or a biologically active fragment or other variant thereof.

6. The construct of claim 5, wherein the biologically active fragment is residues 7-78 of SEQ ID NO:1.

7. The construct of claim 5, wherein the biologically active variant is a sequence that is at least 80% identical to SEQ ID NO:1 or to residues 7-78 of SEQ ID NO:1.

8. The construct of any of claims 2-7, wherein the Tcf site is identical to SEQ ID NO:13.

9. The construct of any of claims 1-8, wherein the second sequence comprises SEQ ID NO:2 or a biologically active fragment or other variant thereof.

10. The construct of claim 9, wherein the biologically active fragment is residues 7-190 of SEQ ID NO:2.

11. The construct of claim 9, wherein the biologically active variant is a sequence that is at least 80% identical to SEQ ID NO:2 or to residues 7-190 of SEQ ID NO:2.

12. The construct of any of claims 1-11, further comprising a multi-cloning site.

13. A kit comprising the construct of claim 12 and instructions for use.

14. The construct of any of claims 1-12, further comprising a promoter, wherein the promoter is operably linked to the enhancer.

15. The construct of claim 14, wherein the promoter is a cell-type specific promoter.

16. The construct of claim 14, wherein the promoter is active in response to an environmental condition.

17. The construct of claim 15 or claim 16, wherein the promoter is selectively active in a cancerous cell.

18. The construct of claim 14, wherein the promoter is a Reg1A promoter or a promoter selected from tyrosinase, prostate-specific antigen (PSA), prostate-specific membrane antigen (PSMA), probasin, human glandular kallikrein (hK2), glial fibrillary acidic protein (GFAP), myelin basic protein (MBP), myelin proteolipid protein, neural specific enolase, neuronal specific synapsin I, Ncx/Hox IILI, albumin, surfactant protein B, thyroglobulin, ovarian-specific promoter, telomerase, CEA, alpha feto protein (AFP), Erb B2, DF3/MUC1, osteocalcin, L-plastin, midkine, secretory leukoprotease inhibitor (SLP1), alpha lactalbumin, Myc-max, somatostatin, Cox2, ornithine decarboxylase, epithelial glyocoprotein 2 (EPG2), c-Myb-responsive promoters, gastrin-releasing peptide, metallothionein, calponin, H19, Tcf, calretinin, calcitonin/calcitonin gene-related peptide, cyclinA, endoglin, IGF-1-R, and E2F-1 promoters.

19. The construct of any of claims 14-18, further comprising a sequence of interest.

20. The construct of claim 19, wherein the sequence of interest, when transcribed in a host cell, produces a therapeutic RNA.

21. The construct of claim 20, wherein the therapeutic RNA mediates RNAi.

22. The construct of claim 19, wherein the sequence of interest, when transcribed and translated in a host cell, produces a therapeutic peptide or protein.

23. The construct of claim 22, wherein the peptide or protein is a toxin.

24. A vector comprising the construct or any of claim 1-12 or 14-23.

25. The vector of claim 24, wherein the vector is a plasmid or viral vector.

26. The vector of claim 25, wherein the viral vector is a recombinant adenovirus.

27. The vector of claim 25, wherein the viral vector is a conditionally replicative oncolytic adenovirus.

28. A host cell comprising the vector of any of claims 24-27.

29. A kit comprising the construct of any of claim 1-12 or 14-23, the vector of any of claims 24-27, or the host cell of claim 28, and instructions for use.

30. A pharmaceutical composition comprising the vector of any of claims 24-27.

31. The pharmaceutical composition of claim 30, wherein the composition is formulated for intravenous, topical, or intra-tumoral administration.

32. A method of treating a patient who needs gene therapy, the method comprising
(a) identifying a patient in need of treatment; and
(b) administering to the patient a therapeutically effective amount of the composition of claim 30 or claim 31, wherein the promoter is selectively active in a cell of the type that needs the therapy within the patient, and the response element is selected among any response element that participates in a pathway that is active in a cell of the type that needs the therapy within the patient.

33. The method of claim 32, wherein the patient suffers from cancer and the promoter of the composition is selectively active in a cell of the type that is cancerous within the patient.

34. The method of claim 32, wherein the patient is a human patient.

35. The method of claim 33 or claim 34, wherein the cancer is gastric cancer.

36. The method of claim 33 or claim 34, wherein the cancer is melanoma, glioma, SCLC, neuroblastoma, hepatocellular carcinoma, lung cancer, and thyroid carcinomas, or the cancer is in a tissue selected from melanocytes, prostate, vascular endothelium, glial, astrocytes, neuronal, neural crest derived cells, liver, type II alveolar and bronchial cells, thyroid, and ovarian, and the promoter is a promoter that is active in a cell of that type and selected from tyrosinase, prostate-specific antigen (PSA), prostate-specific membrane antigen (PSMA), probasin, human glandular kallikrein (hK2), glial fibrillary acidic protein (GFAP), myelin basic protein (MBP), myelin proteolipid protein, neural specific enolase, neuronal specific synapsin I, Ncx/Hox IILI, albumin, surfactant protein B, thyroglobulin, and ovarian-specific promoters.

37. The method of claim 33 or claim 34, wherein the cancer is selected from lung, colon, ovarian, bladder, cervical, liver, glioma, colorectal, pancreatic, cholangiocarcinoma, breast, hepatoma, prostate, brain, osteoblasts, fibrosarcoma, embryonal carcinoma; Wilm's tumours, neuroblastoma, oespohageal, oropharyngeal, endometrial, malignant melanoma of soft parts, gastrointestinal, carcinomas, hematopoietic tumours, soft tissue and bone tumours, mesathelioma, thyroid/thyroid medullary cancer, melanoma, endothelial cells, tumours mutant for p53, cMyb or EWS/WT1, and glioma, and the promoter is a promoter that is active in that type of cancer and selected from telomerase, CEA, alpha feto protein (AFP), Erb B2, DF3/MUC1, osteocalcin, L-plastin, midkine, secretory leukoprotease inhibitor (SLP1), alpha lactalbumin, Myc-max, somatostatin, Cox2, ornithine decarboxylase, epithelial glyocoprotein 2 (EPG2), c-Myb-responsive promoters, gastrin-releasing peptide, metallothionein, calponin, H19, Tcf, calretinin, calcitonin/calcitonin gene-related peptide, cyclinA, endoglin, IGF-1-R, and E2F-1 promoters.
